(19) United States
(12) Patent Application Publication
(10) Pub. No.: US 2018/0177553 A1
(43) Pub. Date: Jun. 28, 2018

(54) SYSTEM AND METHOD FOR OPTIMIZING AN IMPLANT POSITION IN AN ANATOMICAL JOINT

(71) Applicant: Episurf IP-Management AB, Stockholm (SE)

(72) Inventors: Anders Karlsson, Kavlinge (SE); Richard Lilliestråle, Stockholm (SE)

(21) Appl. No.: 15/853,605

(22) Filed: Dec. 22, 2017

Related U.S. Application Data
(63) Continuation of application No. PCT/EP2016/082455, filed on Dec. 22, 2016.

(51) Int. Cl. A61B 34/10; A61F 2/32; A61F 2/38; A61F 2/40; A61F 2/42; G06T 19/20

(52) U.S. Cl. CPC A61B 34/10; A61F 2/32; A61F 2/38; A61F 2/40; A61F 2/42; G06T 7/0012; A61B 2034/105; A61B 2034/108; A61B 2034/102; A61B 2034/107; G06T 2219/2004; G06T 19/20

(57) ABSTRACT

In accordance with one or more embodiments herein, a system for optimizing an implant position in an anatomical joint of a patient is provided. The system comprises a storage media and a processor which is configured to: receive medical image data representing a three dimensional image of the joint from the storage media; obtain a three dimensional virtual model of the joint which is based on the received medical image data; identify damage in the joint based on the received medical image data and/or the three dimensional virtual model of the joint; select a suitable implant template based on the identified damage; and position the selected implant template in the three dimensional virtual model of the joint. The processor is configured to position the implant template by: placing the implant template so that at least a major part of the damage is covered; and optimizing the tilt of the implant axis in order to minimize the total penetration into the bone.

### CROSS-REFERENCE TO RELATED APPLICATION

This application is a continuation of PCT Application No. PCT/EP2016/082455, filed Dec. 22, 2016, entitled “SYSTEM AND METHOD FOR OPTIMIZING AN IMPLANT POSITION IN AN ANATOMICAL JOINT,” the content of which is incorporated by reference herein.

### TECHNICAL FIELD

The present disclosure relates generally to systems and methods for optimizing an implant position in an anatomical joint of a patient.

### BACKGROUND

Surgical implants have since long been used for cartilage repair in anatomical joints. For a long time, a surgeon wishing to use an implant for cartilage repair could only choose from a selection of ready-made implants, and try to fit these in the best possible way, but over the years, various ways of customizing the implants to the individual patient have been developed, as shown e.g. in WO 2016/005542 and US 2007/0276224.

### Problems with the Prior Art

Even though individually customized implants have been used for some time, the positioning of these customized implants has not developed much from the time of the ready-made implants. Even though it is now possible to customize the implant surface, an implant is still generally positioned with its axis tangential to the cartilage surface, as shown e.g. in WO 2016/005542. The advantage of this is that the thickness of the implant usually becomes relatively uniform. However, the positioning of the implant may not be optimal in other respects.

There is a need to address these problems of conventional methods and systems.

### SUMMARY

The above described problems are addressed by the claimed system for optimizing an implant position in an anatomical joint of a patient. The claimed system comprises a storage media and a processor which is configured to: receive medical image data representing a three dimensional image of the joint from the storage media; obtain a three dimensional virtual model of the joint which is based on the received medical image data; identify damage in the joint based on the received medical image data and/or the three dimensional virtual model of the joint; select a suitable implant template based on the identified damage; and position the selected implant template in the three dimensional virtual model of the joint. The processor is configured to position the implant template by: placing the implant template so that at least a major part of the damage is covered; and optimizing the tilt of the implant axis in order to minimize the total penetration into the bone. This makes the implanting less invasive, which is an advantage in many respects, e.g. if the need arises for subsequent implants.

In embodiments, the processor is configured to position the implant template by placing the implant template so that the implant hat will at all points be thick enough to ensure mechanical stability, and preferably also thick enough to ensure firm anchoring towards cartilage and bone. The processor may e.g. be configured to position the implant template by placing the implant template so that the implant hat at each point of its circumference penetrates at least a predetermined minimum depth Dₘᵢₙ into the bone. This ensures that the whole of the implant hat will have at least a minimum thickness, and will thus not easily break.

In embodiments, the processor is configured to optimize the tilt of the implant axis by minimizing the maximum penetration depth Dₘₐₓ into the bone along the circumference of the implant hat. This ensures that the hole to be drilled in the bone will not become deeper than necessary.

In embodiments, the processor is configured to optimize the tilt of the implant axis by minimizing the total volume of bone and/or cartilage to be removed for implanting the implant. This optimization is especially advantageous when implanting a combined implant having more than one implant axis.

In embodiments, the processor is configured to optimize the tilt of the implant axis by minimizing the surface area of the implant penetration into the bone. The surface area may e.g. be determined by multiplying the average depth of the hole to be drilled in the bone by the circumference.

In embodiments, the processor is configured to restart the optimization of the implant position if at least one predetermined demand on the implant design is not fulfilled. One such predetermined demand on the implant design may be that the implant hat must have a predetermined minimum thickness Tₘᵢₙ.

The above described problems are also addressed by the claimed method of optimizing an implant position in an anatomical joint of a patient. The claimed method comprises: receiving medical image data representing a three dimensional image of the joint; obtaining a three dimensional virtual model of the joint which is based on the received medical image data; identifying damage in the joint based on the received medical image data and/or the three dimensional virtual model of the joint; selecting a suitable implant template based on the identified damage; and positioning the selected implant template in the three dimensional virtual model of the joint. The positioning involves: placing the implant template so that at least a major part of the damage is covered; and optimizing the tilt of the implant axis in order to minimize the total penetration into the bone. This makes the implanting less invasive, which is an advantage in many respects, e.g. if the need arises for subsequent implants.

In embodiments, the positioning involves placing the implant template so that the implant hat will at all points be thick enough to ensure mechanical stability, and preferably also thick enough to ensure firm anchoring towards cartilage and bone. The positioning may e.g. involve placing the implant template so that the implant hat at each point of its circumference penetrates at least a predetermined minimum depth Dₘᵢₙ into the bone. This ensures that the whole of the implant hat will have at least a minimum thickness, and will thus not easily break.

In embodiments, the optimization of the tilt of the implant axis involves minimizing the maximum penetration depth Dₘₐₓ into the bone along the circumference of the implant hat. This ensures that the hole to be drilled in the bone will not become deeper than necessary.

In embodiments, the optimization of the tilt of the implant axis involves minimizing the total volume of bone and/or cartilage to be removed for implanting the implant. This optimization is especially advantageous when implanting a combined implant having more than one implant axis.

In embodiments, the optimization of the tilt of the implant axis involves minimizing the surface area of the implant penetration into the bone. The surface area may e.g. be determined by multiplying the average depth of the hole to be drilled in the bone by the circumference.

In embodiments, the optimization of the implant position is restarted if at least one predetermined demand on the implant design is not fulfilled. One such predetermined demand on the implant design may be that the implant hat must have a predetermined minimum thickness Tₘᵢₙ.

The anatomical joint may e.g. be a knee, an ankle, a hip, a toe, an elbow, a shoulder, a finger or a wrist.

The above described problems are also addressed by a non-transitory machine-readable medium on which is stored machine-readable code which, when executed by a processor, controls the processor to perform any one of the above described methods.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 shows a schematic view of a system for optimizing an implant position in an anatomical joint of a patient, in accordance with one or more embodiments described herein.

FIG. 2 is a flow diagram for a method for optimizing an implant position in an anatomical joint, in accordance with one or more embodiments described herein.

FIG. 3 shows an image of a three dimensional virtual model of a joint where the maximum and minimum penetration depths of an implant at a selected axis tilt have been indicated.

FIG. 4 shows an image of a three dimensional virtual model of a joint where the maximum and minimum penetration depths of an implant with two axes at selected axis tilts have been indicated.

FIGS. 5a and 5b show schematic examples of different potential implant positions in an anatomical joint, in accordance with one or more embodiments described herein.

FIGS. 6a and 6b show other schematic examples of different potential implant positions in an anatomical joint, in accordance with one or more embodiments described herein.

FIGS. 7a and 7b show yet other schematic examples of different potential implant positions in an anatomical joint, in accordance with one or more embodiments described herein.

FIGS. 8a and 8b show examples of different potential implant positions in an anatomical joint, in accordance with one or more embodiments described herein.

FIG. 9 shows a flow diagram exemplifying the steps from the capturing of radiology image data to the design and production of a medical implant, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

### Introduction

The present disclosure relates generally to systems and methods for optimizing a position of an implant for cartilage repair in an anatomical joint of a patient. Optimization of implant position may be especially important for implants to be positioned on concave surfaces, such as trochlea implants, since on concave surfaces, the implant angle may be selected in many different ways.

In some embodiments, the anatomical joint is a knee, but the methods and systems presented herein may be used for optimizing the implant position in any suitable anatomical joint, e.g. an ankle, a hip, a toe, an elbow, a shoulder, a finger or a wrist. In a non-limiting example, the implant is intended to be positioned in the trochlea of an anatomical joint.

The medical image data may be retrieved directly from a digital imaging and communications in medicine (DICOM) file or any other suitable image file format. The DICOM format, or a comparable image file format, is advantageous for visualizing different parts of the anatomical joint. The DICOM format, or a comparable image file format, may be used for visualizing different parts of a knee, such as the femoral condyles and the trochlea area, or different parts of any other relevant anatomical joint that is being investigated, such as the talus of the ankle.

The three dimensional virtual model of the joint may be obtained in many different ways. It may e.g. be obtained from a storage media, or be generated based on a series of radiology images captured during a process of scanning radiology images through different layers of the anatomical joint or part of it, which captures all the radiology image data necessary to generate a three dimensional virtual model of the anatomical joint or part of it in an image segmentation process based on said radiology images. The three dimensional virtual model may be used for visualizing tissues, bone, cartilage and damages in relation to femoral knee bone and cartilage, or bone and cartilage of any other relevant anatomical joint that is being investigated.

The processor may in some embodiments comprise several different processors which together perform the claimed functions. In the same way, the storage media may in some embodiments comprise several different storage media which together perform the claimed functions.

The implant axis is the axis running through the center of the circular implant hat. The implant axis normally coincides with the axis of the implant peg, which fixates the implant.

System and method embodiments of the disclosed solution are presented in more detail in connection with the figures.

### System Architecture

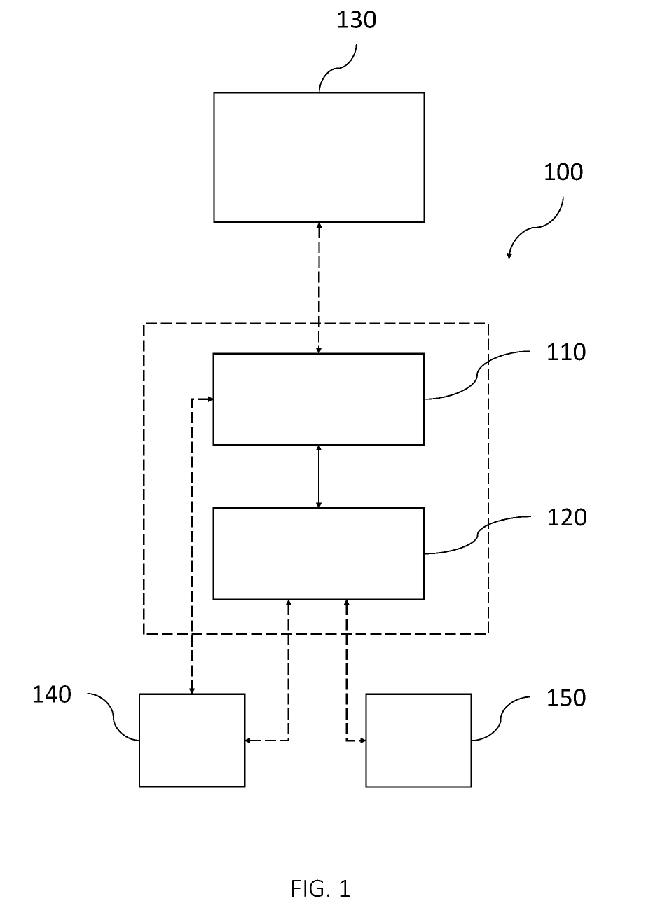

FIG. 1 shows a schematic view of a system 100 for optimizing an implant position in an anatomical joint of a patient. According to embodiments, the system 100 comprises a storage media 110, configured to receive and store image data and parameters. In some embodiments, the system 100 is communicatively coupled, as indicated by the dashed arrow in FIG. 1, to an imaging system 130. The imaging system 130 may be configured to capture or generate radiology images, such as for example X-ray images, ultrasound images, computed tomography (CT) images, nuclear medicine including positron emission tomography (PET) images, and magnetic resonance imaging (MRI) images. The storage media 110 may be configured to receive and store medical image data from the imaging system 130.

According to embodiments, the system 100 further comprises a processor 120, which may for example be a general data processor, or other circuit or integrated circuit capable of executing instructions to perform various processing operations.

In one or more embodiments, the processor 120 is configured to:

receive medical image data representing a three dimensional image of the joint from the storage media 110;

obtain a three dimensional virtual model of the joint which is based on the received medical image data;

identify damage in the joint based on the received medical image data and/or the three dimensional virtual model of the joint;

select a suitable implant template based on the identified damage; and

position the selected implant template in the three dimensional virtual model of the joint;

wherein the processor 120 is configured to position the implant template by:

placing the implant template so that at least a major part of the damage is covered; and

optimizing the tilt of the implant axis A in order to minimize the total penetration into the bone.

The medical image data may for example be captured during a process of scanning radiology images through different layers of the anatomical joint or part of it, which captures all the radiology image data necessary to generate a three-dimensional virtual model of the anatomical joint or part of it in an image segmentation process based on the medical image data.

The damage may be identified automatically by the processor 120, or be identified manually by an operator of the system with the help of the processor 120. In some embodiments, damage may be identified for both bone parts and cartilage parts of the anatomical joint. Alternatively, damage to bone parts only, or damage to cartilage parts only, or damage to other tissue parts such as tendons or ligaments, may be identified, depending on the application.

The implant template may be selected automatically by the processor 120, or be selected manually by an operator of the system with the help of the processor 120. In embodiments, the processor 120 may be configured to select a suitable implant template from a predefined set of implant templates with varying dimensions. In this context, a suitable implant template means an implant template having a type and dimensions that match the identified damage, thereby making it suitable for repairing the identified damage.

The selected implant template may be positioned in the three dimensional virtual model of the joint automatically by the processor 120, or be positioned in the three dimensional virtual model of the joint manually by an operator of the system with the help of the processor 120. In embodiments, the processor 120 proposes alternative positions from which the operator of the system can choose. It may also be possible for the operator of the system to manually adjust an automatically generated implant template position.

The processor 120 does not necessarily position the implant template to cover all the identified damage—as long as a major part of the damage is covered, the implant may still be used for cartilage repair. It is sometimes not desirable to use an implant which is large enough to cover all the identified damage, since this may lead to the unnecessary removal of healthy bone and/or cartilage when the identified damage does not have the same shape as the implant template.

The processor 120 may be configured to position the implant template by placing the implant template so that the implant hat H will at all points be thick enough to ensure mechanical stability, and preferably also thick enough to ensure firm anchoring towards cartilage and bone. If the hat H of the final implant is too thin in certain points, the risk of it breaking may be too high. Further, if the hat H of the final implant is not thick enough to ensure firm anchoring towards cartilage and bone, there may be a risk of liquid penetrating the surface under the hat H, so that the implant may be loosened. The mechanical stability and/or the firm anchoring may e.g. be ensured by the processor being configured to place the implant template so that the implant hat H at each point of its circumference penetrates at least a predetermined minimum depth Dₘᵢₙ into the bone. Dₘᵢₙ may be any suitable depth, such as e.g. 2 or 3 mm. Dₘᵢₙ may also be 0 mm, if it is enough that the implant hat H at least touches the bone along its whole circumference. Since the cartilage has a certain thickness, it may be enough to ensure that the implant hat H at least touches the bone along its whole circumference in order to ensure that the implant hat H has at least a minimum thickness at all points.

The processor 120 may optimize the tilt of the implant axis A in many different ways in order to minimize the total penetration into the bone.

The processor 120 may optimize the tilt of the implant axis A by minimizing the maximum penetration depth Dₘₐₓ into the bone along the circumference of the implant hat H. This ensures that the hole to be drilled in the bone will not become deeper than necessary.

The processor 120 may optimize the tilt of the implant axis A by minimizing the total volume of bone and/or cartilage to be removed for implanting the implant. This optimization is especially advantageous when implanting a combined implant having more than one implant axis, or when implanting an implant in a concave surface of a joint. Even if the implant in some points penetrates deep into the bone, the total volume to be drilled away may be smaller if the tilt of the implant axis A is optimized by minimizing the total volume of bone and/or cartilage to be removed for implanting the implant.

The processor 120 may optimize the tilt of the implant axis A by minimizing the surface area of the implant penetration into the bone. The surface area may e.g. be determined by multiplying the average depth of the hole to be drilled in the bone by the circumference.

In embodiments, the processor 120 is configured to restart the optimization of the implant position if at least one predetermined demand on the implant design is not fulfilled. One such predetermined demand on the implant design may be that the implant hat H must have a predetermined minimum thickness Tₘᵢₙ.

In one or more embodiments, the system 100 may optionally comprise a display 140 configured to display image data. The display 140 may be configured to receive image data for display via the processor 120, and/or to retrieve image data for display directly from the storage media 110, possibly in response to a control signal received from the processor 120 or an inputter 150, which is further presented below. The processor 120 may be configured to visualize the identified damage in the three dimensional virtual model of the joint using the display 140.

In some embodiments, the system 100 may further optionally comprise one or more inputters 150 configured to receive user input. The inputter 150 is typically configured to interpret received user input and to generate control signals in response to said received user input. The display 140 and the inputter 150 may be integrated in, connected to or communicatively coupled to the system 100. The inputter 150 may for instance be configured to interpret received user input that is being input in connection with a displayed virtual model, and generate control signals in response to said received user input, to trigger display of an image or manipulation of image data being displayed, wherein the manipulations may be temporary or permanent. Such manipulations may for example include providing annotations, moving or changing an image or part of an image, changing the viewing perspective, zooming in or out, and/or any other suitable form of manipulation that enables the user to view and analyze the displayed image data in an improved manner. An inputter 150 may for example comprise a selection of a keyboard, a computer mouse, one or more buttons, touch functionality, a joystick, and/or any other suitable input device. In some embodiments, the processor 120 may be configured to receive a control signal from the inputter 150 and to process image data that is being displayed using display 140, or in other words manipulate a displayed image, in response to the received control signal.

In some embodiments, the anatomical joint is a knee. In other embodiments, the anatomical joint may be any other anatomical joint suitable for damage determination using image data analysis, such as an ankle, a hip, a toe, an elbow, a shoulder, a finger or a wrist.

The processor 120 may be configured to perform the method steps of any or all of the embodiments presented herein.

### Method Embodiments

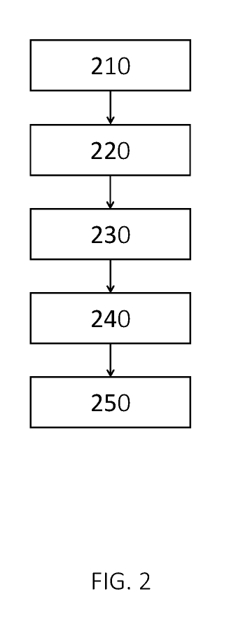

FIG. 2 is a flow diagram for a method for optimizing an implant position in an anatomical joint. In accordance with one or more to embodiments, the method comprises:

In step 210: receiving medical image data representing a three dimensional image of the joint.

The medical image data may for example be captured during a process of scanning radiology images through different layers of the anatomical joint or part of it, which captures all the radiology image data necessary to generate a three-dimensional virtual model of the anatomical joint or part of it in an image segmentation process based on the medical image data.

In some embodiments, the anatomical joint is a knee. In other embodiments, the anatomical joint may be any other anatomical joint suitable for damage determination using image data analysis, such as an ankle, a hip, a toe, an elbow, a shoulder, a finger or a wrist.

In step 220: obtaining a three dimensional virtual model of the joint which is based on the received medical image data.

In step 230: identifying damage in the joint based on the received medical image data and/or the three dimensional virtual model of the joint.

The damage may be identified automatically or manually. In some embodiments, damage may be identified for both bone parts and cartilage parts of the anatomical joint. Alternatively, damage to bone parts only, or damage to cartilage parts only, or damage to other tissue parts, may be identified, depending on the application. It may in some embodiments be advantageous to analyze both bone and cartilage of the depicted joint in the input medical image data, as the combination of the two may provide additional information, but all embodiments described herein may also be performed when only one of the substances bone and cartilage, or any other tissue part, of the depicted joint is analyzed.

In one or more embodiments, method step 230 comprises detecting that the intensity in an area within or adjacent to the bone and/or cartilage parts of the anatomical joint is higher or lower than a predetermined threshold. Depending on the settings of the imaging device that has captured the analyzed medical image data, the analyzed image may for example represent the following substances with different intensity levels: cortical bone, liquids, cartilage, fat/bone marrow and meniscus. Different intensity levels in the analyzed image correspond to different signal intensity levels and these may typically be represented by pixel/voxel values ranging from 0 to 1, or in a visual representation shown as grey scale levels from white to black. In embodiments where the pixel/voxel values range from 0 to 1, a predetermined threshold is set to a suitable value between 0 and 1, or in other words to a suitable grey scale value.

In one or more embodiments, method step 230 further, or alternatively, comprises detecting an irregular shape of a contour of the at least one tissue part of the anatomical joint and determining whether this represents a damage to the anatomical joint.

In one or more embodiments, method step 230 comprises marking, visualizing or in another way indicating the identified damage to the anatomical joint. Marking, visualizing, or indicating the identified damage may include changing the pixel/voxel value of one or more pixels/voxels on, in connection with, or surrounding a pixel/voxel identified to belong to identified damage, such that the identified damage is visually distinguished and noticeable to a user/viewer. Such a change of pixel/voxel values of one or more pixels/voxels on, in connection with, or surrounding a pixel/voxel identified to belong to identified damage may for example comprise a selection of the following:
changing the luminance/intensity values of one or more pixels/voxels identified as being located on identified damage;
changing one or more chrominance/color values of one or more pixels/voxels identified as being located on identified damage;
changing the luminance/intensity values of one or more pixels/voxels identified as surrounding identified damage;
changing one or more chrominance/color values of one or more pixels/voxels identified as surrounding identified damage; and/or
adding an annotation, symbol or other damage indicator to the image, in connection with one or more pixels/voxels identified as being located on, or surrounding, identified damage.

In step 240: selecting a suitable implant template based on the identified damage.

The implant template may be selected automatically or manually. In embodiments, a suitable implant template may be selected from a predefined set of implant templates with varying dimensions. In this context, a suitable implant template means an implant template having a type and dimensions that match identified damage, thereby making it suitable for repairing the identified damage.

In step 250: positioning the selected implant template in the three dimensional virtual model of the joint.

The selected implant template may be positioned automatically or manually. It may also be possible for the operator of the system to manually adjust an automatically generated implant template position.

In different embodiments, the positioning in step 250 may involve:
placing the implant template so that at least a major part of the damage is covered; and
optimizing the tilt of the implant axis A in order to minimize the total penetration into the bone.

The implant template does not have to cover all the identified damage—as long as a major part of the damage is covered, the implant may still be used for cartilage repair. It is sometimes not desirable to use an implant which is large enough to cover all the identified damage, since this may lead to the unnecessary removal of healthy bone and cartilage when the identified damage does not have the same shape as the implant template.

The positioning may involve placing the implant template so that the implant hat H will at all points be thick enough to ensure mechanical stability, and preferably also thick enough to ensure firm anchoring towards cartilage and bone. If the hat H of the final implant is too thin in certain points, the risk of it breaking may be too high. Further, if the hat H of the final implant is not thick enough to ensure firm anchoring towards cartilage and bone, there may be a risk of liquid penetrating the surface under the hat H, so that the implant may be loosened. The mechanical stability and/or the firm anchoring may e.g. be ensured by the positioning involving placing the implant template so that the implant hat H at each point of its circumference penetrates at least a predetermined minimum depth Dₘᵢₙ into the bone. Dₘᵢₙ may be any suitable depth, such as e.g. 2 or 3 mm. Dₘᵢₙ may also be 0 mm, if it is enough that the implant hat H at least touches the bone along its whole circumference. Since the cartilage has a certain thickness, it may be enough to ensure that the implant hat H at least touches the bone along its whole circumference in order to ensure that the implant hat H has at least a minimum thickness at all points.

The tilt of the implant axis A may be optimized in many different ways in order to minimize the total penetration into the bone.

The tilt of the implant axis A may e.g. be optimized by minimizing the maximum penetration depth Dₘₐₓ into the bone along the circumference of the implant hat H. This ensures that the hole to be drilled in the bone will not become deeper than necessary.

The tilt of the implant axis A may e.g. be optimized by minimizing the total volume of bone and/or cartilage to be removed for implanting the implant. This optimization is especially advantageous when implanting a combined implant having more than one implant axis, or when implanting an implant in a concave surface of a joint. Even if the implant in some points penetrates deep into the bone, the total volume to be drilled away may be smaller if the tilt of the implant axis A is optimized by minimizing the total volume of bone and/or cartilage to be removed for implanting the implant.

The tilt of the implant axis A may e.g. be optimized by minimizing the surface area of the implant penetration into the bone. The surface area may e.g. be determined by multiplying the average depth of the hole to be drilled in the bone by the circumference.

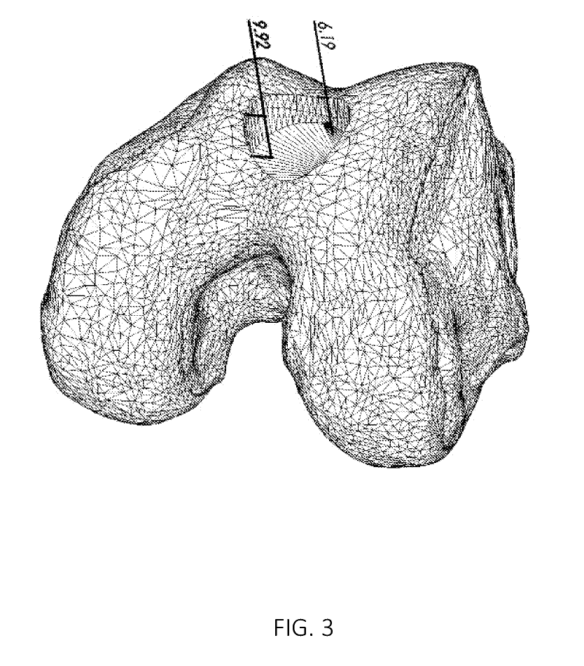

When the tilt of the implant axis A is optimized, the penetration into the bone may be visualized. FIG. 3 shows an image of a three dimensional virtual model of a joint where the maximum and minimum penetration depths of an implant at a selected axis tilt have been indicated. This visualization makes it easier to manually optimize the tilt of the implant axis A to minimize the total penetration into the bone. When the tilt of the implant axis A is optimized automatically, the visualization is not necessary, but may be used to double-check the automated procedure.

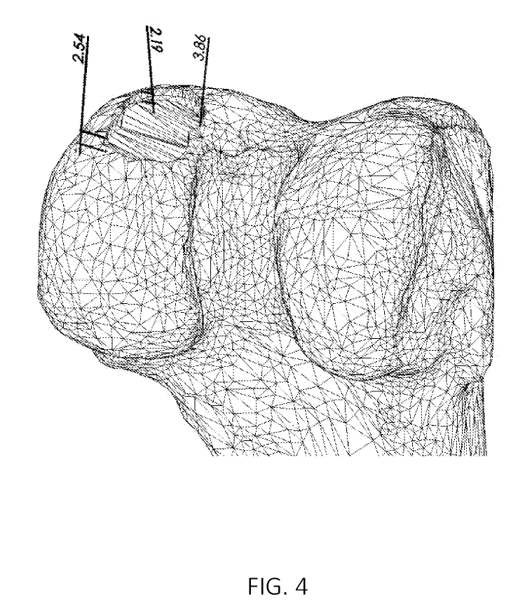

For implants having two implant axes, so called twin implants, the optimization may be more complicated. Preferably, the tilt of each of the implant axes is optimized separately to minimize the total penetration into the bone for each part of the twin implant. The implant parts are then joined together to form a twin implant. FIG. 4 shows an image of a three dimensional virtual model of a joint where the maximum and minimum penetration depths for each part of an implant having two axes at selected axis tilts have been indicated. This visualization makes it easier to manually optimize the tilt of the implant axes to minimize the total penetration into the bone, and to ensure that the implant parts fit together to form a twin implant. When the tilt of the implant axes is optimized automatically, the visualization is not necessary, but may be used to double-check the automated procedure.

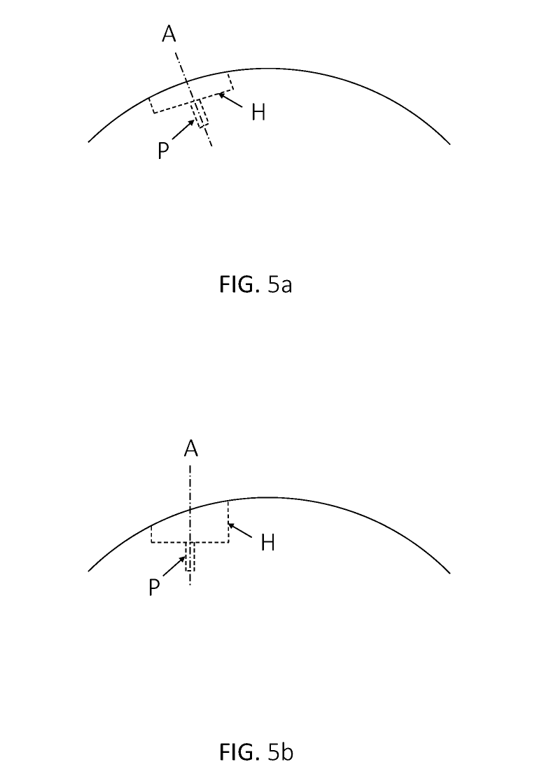

FIGS. 5a and 5b show schematic examples of different potential implant positions in an anatomical joint, in accordance with one or more embodiments described herein. More specifically, FIG. 5a shows an implant with an implant hat H and an implant peg P, positioned so that the implant axis A is substantially parallel to the surface normal at the center of the implant, while FIG. 5b shows an implant positioned in a different way. The positioning according to FIG. 5a may minimize the average penetration depth or the maximum penetration depth, but may not minimize the total volume of bone and/or cartilage to be removed for implanting the implant or the surface area of the implant penetration into the bone.

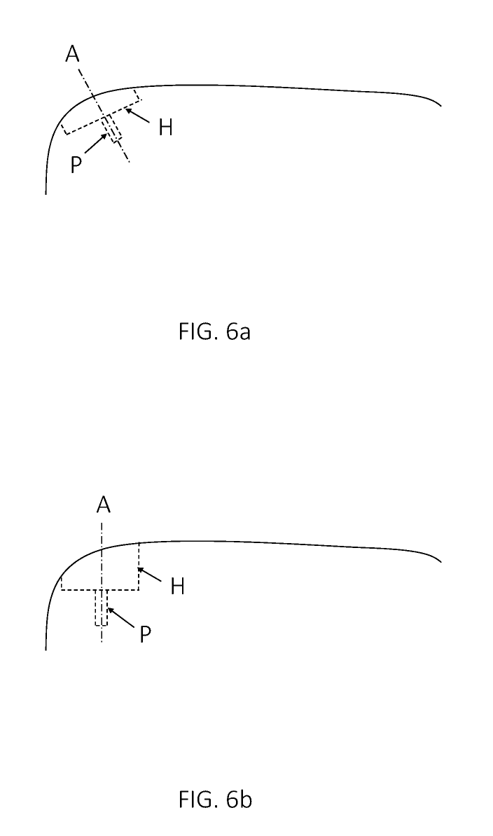

FIGS. 6a and 6b show other schematic examples of different potential implant positions in an anatomical joint, in accordance with one or more embodiments described herein. More specifically, FIG. 6a shows an implant with an implant hat H and an implant peg P, positioned so that the implant axis A is substantially parallel to the surface normal at the center of the implant, while FIG. 6b shows an implant positioned in a different way. The positioning according to FIG. 6a may minimize the average penetration depth or the maximum penetration depth, but may not minimize the total volume of bone and/or cartilage to be removed for implanting the implant or the surface area of the implant penetration into the bone.

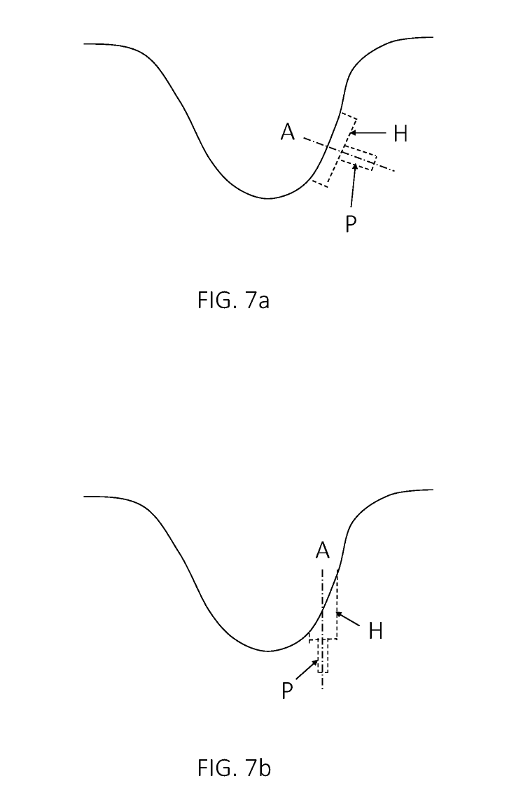

FIGS. 7a and 7b show yet other schematic examples of different potential implant positions in an anatomical joint, in accordance with one or more embodiments described herein. More specifically, FIG. 7a shows an implant with an implant hat H and an implant peg P, positioned so that the implant axis A is substantially parallel to the surface normal at the center of the implant, while FIG. 7b shows an implant positioned in a different way. When the implant is to be positioned in a joint having the shape shown in FIGS. 7a and 7b, the implant position may be more optimized if the total volume of bone and/or cartilage to be removed for implanting the implant or the surface area of the implant penetration into the bone is minimized than if the maximum penetration depth Dₘₐₓ into the bone along the circumference of the implant hat H is minimized.

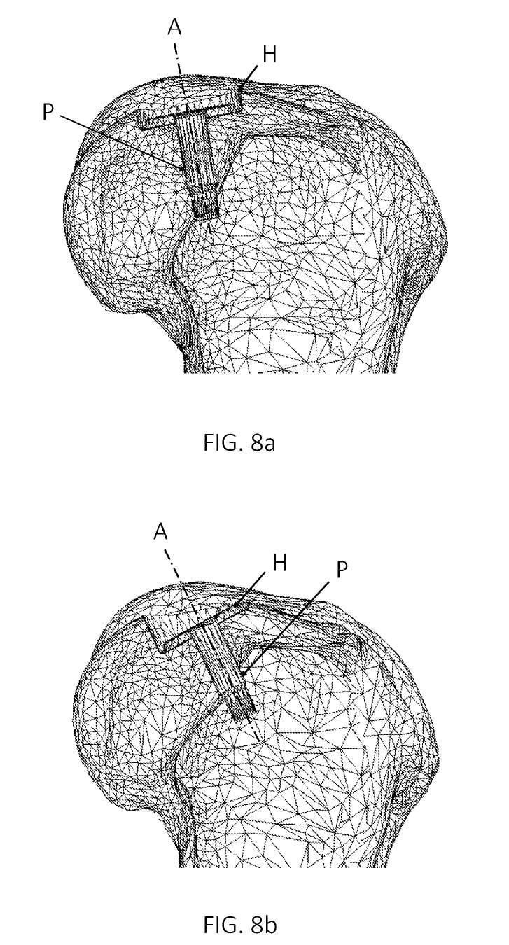

FIGS. 8a and 8b show examples of different potential implant positions in an anatomical joint, in accordance with one or more embodiments described herein. More specifically, FIG. 8a shows an implant with an implant hat H and an implant peg P, positioned so that the implant axis A is almost parallel to the surface normal at the center of the implant, while FIG. 8b shows an implant positioned in a different way. The positioning according to FIG. 8a may minimize the average penetration depth or the maximum penetration depth, but may not minimize the total volume of bone and/or cartilage to be removed for implanting the implant or the surface area of the implant penetration into the bone.

### Use Case Embodiment

To set the presently disclosed methods and systems in a larger context, the method of optimizing an implant position in an anatomical joint of a patient according to any of the disclosed embodiments may in use case embodiments be preceded by capturing and/or obtaining medical image data representing an anatomical joint, and may further be followed by the design and production of a medical implant.

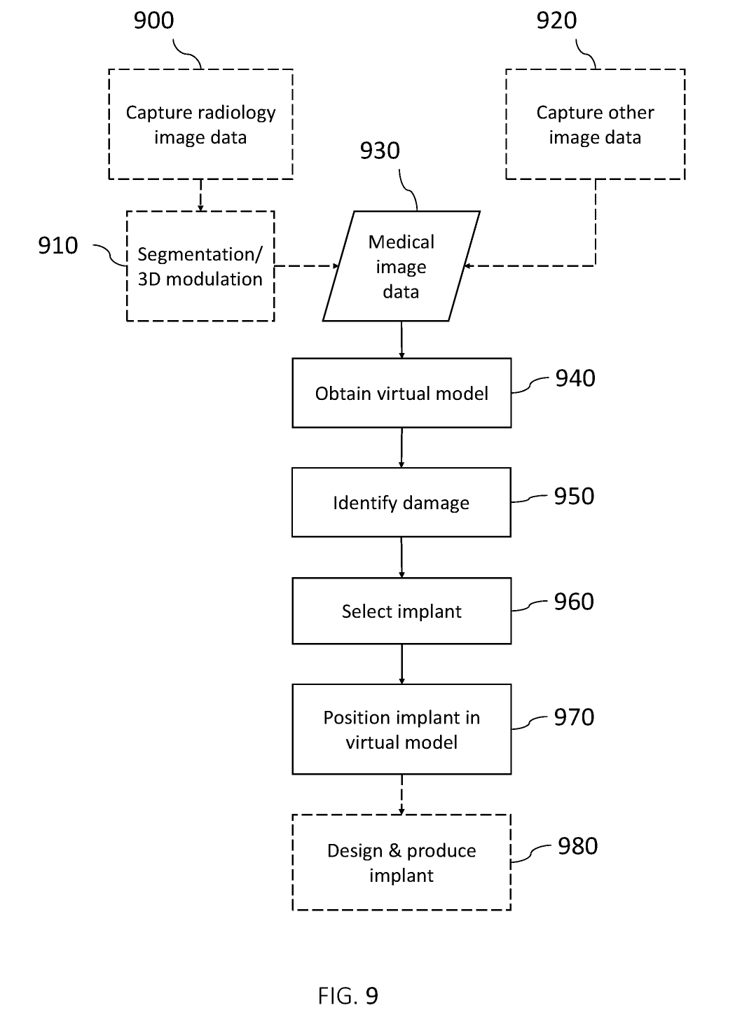

FIG. 9 is a flow diagram exemplifying one such larger context, including receiving radiology/medical image data from an image source, obtaining a three dimensional virtual model of the joint, identifying damage in the joint, selecting a suitable implant template based on the identified damage, and positioning the selected implant template in the virtual model, in accordance with one or more embodiments described herein. FIG. 9 further includes steps of designing and producing a medical implant suitable for repairing identified damage in an anatomical joint. In FIG. 9, everything except the receiving of medical image data from an image source, obtaining a three dimensional virtual model of the joint, identifying damage in the joint, selecting a suitable implant template based on the identified damage and positioning the selected implant template in the virtual model is marked with dashed lines to clarify that they are optional steps shown in the figure to provide context only, and not essential to any of the embodiments presented herein. Especially, step 980 relating to the design and production of the medical implant is not part of the embodiments presented herein.

According to the example shown in FIG. 9, medical image data 930 may be obtained in a step 900 in the form of radiology image data from a radiology imaging system. The radiology image data obtained may for example be generated using one or more of a variety of imaging techniques such as X-ray images, ultrasound images, computed tomography (CT) images, nuclear medicine including positron emission tomography (PET) images, and magnetic resonance imaging (MRI) images. The radiology image data may for example be captured during a process of scanning radiology images through different layers of the anatomical joint or part of it, which captures all the radiology image data necessary to generate a three dimensional virtual model of the anatomical joint or part of it based on the radiology image data.

The image data obtained in step 900 may further be processed in a step 910, by performing segmentation and 3D modulation to obtain a three dimensional virtual model of what is depicted in the captured image data. For instance, if the image data captured depict an anatomical joint, the three dimensional virtual model would be a three dimensional virtual model of the anatomical joint. Medical image data may also be obtained in step 920 from a different kind of image source that provides 2D image data. The three dimensional virtual model and the 2D image data both depict the same object, namely the anatomical joint of interest for damage determination. The 2D image data and the three dimensional virtual model may represent only a part of the anatomical joint.

In a use case embodiment, an operator may use the system as follows. The operator receives 930 medical image data representing a three dimensional image of the joint, and obtains 940 a three dimensional virtual model of the joint based on the received medical image data. The three dimensional virtual model may be generated by the system or retrieved from another source. Damage in the joint is then identified 950 based on the received medical image data and/or the three dimensional virtual model of the joint, either automatically by the system or manually by the operator. Damage in the joint may be identified based on the received medical image data before the three dimensional virtual model of the joint is obtained.

The operator then selects 960 a suitable implant template based on the identified damage. The implant template may be selected automatically by the processor 120, manually by the operator with the help of the processor 120, or entirely manually by the operator. A suitable implant template may e.g. be selected from a predefined set of implant templates with varying dimensions. In this context, a suitable implant template means an implant template having a type and dimensions that match the identified damage, thereby making it suitable for repairing the identified damage. The implant template should have a size essentially corresponding to the size of the identified damage, but may be slightly larger or slightly smaller. If the identified damage has a shape making it unsuitable for repair with a circular implant, a so-called twin implant may e.g. be used instead.

The operator then positions 970 the selected implant template in the three dimensional virtual model of the joint. The positioning may be automated or manual, or partly automated and partly manual, e.g. by the processor 120 proposing a position which can then be adjusted manually by the operator. The system may e.g. automatically propose a position which the operator can then adjust manually. The implant template is first placed so that at least a major part of the damage is covered. Then, the implant axis A is tilted in such a way that the total penetration into the bone is minimized.

During the positioning, the maximum and minimum penetration depths of the implant at selected axis tilts may be visualized, as shown in FIGS. 3 and 4. If the operator positions the selected implant template manually or partly manually, such a visualization makes it easier to see how different axis tilts affect the total penetration into the bone, so that this can be minimized. The operator may try different axis tilts and, preferably in real time, see how they affect the maximum and minimum penetration depths. If the tilt of the implant axis A is optimized automatically, the operator may use such a visualization to check the results of the automated procedure, and verify or adjust the selected implant position.

The visualization shown in FIGS. 8a and 8b may also be used by the operator to verify the selected implant template and implant position. This visualization clearly shows the position of a selected implant template at different selected axis tilts.

When an implant having two implant axes, a so called twin implant, is to be positioned, the operator may position each of the implant parts separately. The visualization shown in FIG. 4 enables the operator to try different axis tilts for the two implant parts, and see how they affect the maximum and minimum penetration depths of the two implant parts. Preferably, the tilt of each of the implant axes is optimized separately, to minimize the total penetration into the bone for each part of the twin implant. The visualization shown in FIG. 4 shows, preferably in real time, the maximum and minimum penetration of both parts of the twin implant at the same time. This visualization makes it easier to manually optimize the tilts of the implant axes to minimize the total penetration into the bone, and to ensure that the implant parts fit together to form a twin implant. If the tilt of the implant axes is optimized automatically, the operator may use such a visualization to check the results of the automated procedure, and verify or adjust the shape of the resulting twin implant and selected implant position.

The operator preferably positions the implant template so that the implant hat H will at all points be thick enough to ensure mechanical stability, and preferably also thick enough to ensure firm anchoring towards cartilage and bone. One way of doing this is to position the implant template deep enough into the bone that the implant hat H at each point of its circumference penetrates at least a predetermined minimum depth Dₘᵢₙ into the bone.

The operator may minimize the total penetration into the bone by tilting the implant axis A in such a way that the implant nowhere along its circumference penetrates unnecessarily deep into the bone, i.e. minimize the maximum penetration depth Dₘₐₓ into the bone along the circumference of the implant hat H. Another way for the operator to minimize the total penetration is to calculate the total volume of bone (and possibly also cartilage) to be removed for implanting the implant, and tilt the implant axis A in such a way that the total volume of bone (and/or cartilage) to be removed for implanting the implant is minimized. Another way is to calculate the surface area of the implant penetration into the bone, and tilt the implant axis A in such a way that the surface area of the implant penetration into the bone is minimized.

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure. Accordingly, the scope of the invention is defined only by the claims.

### Further Embodiments

Where applicable, various embodiments provided by the present disclosure can be implemented using hardware, software, or combinations of hardware and software. Also where applicable, the various hardware components and/or software components set forth herein can be combined into composite components comprising software, hardware, and/or both without departing from the claimed scope of the present disclosure. Where applicable, the various hardware components and/or software components set forth herein can be separated into sub-components comprising software, hardware, or both without departing from the claimed scope of the present disclosure. In addition, where applicable, it is contemplated that software components can be implemented as hardware components, and vice-versa. The method steps of one or more embodiments described herein may be performed automatically, by any suitable processing unit, or one or more steps may be performed manually. Where applicable, the ordering of various steps described herein can be changed, combined into composite steps, and/or separated into sub-steps to provide features described herein.

Software in accordance with the present disclosure, such as program code and/or data, can be stored in non-transitory form on one or more machine-readable mediums. It is also contemplated that software identified herein can be implemented using one or more general purpose or specific purpose computers and/or computer systems, networked and/or otherwise.

In embodiments, there are provided a computer program product comprising computer readable code configured to, when executed in a processor, perform any or all of the method steps described herein. In some embodiments, there are provided a non-transitory computer readable memory on which is stored computer readable and computer executable code configured to, when executed in a processor, perform any or all of the method steps described herein.

In one or more embodiments, there is provided a non-transitory machine-readable medium on which is stored machine-readable code which, when executed by a processor, controls the processor to perform the method of any or all of the method embodiments presented herein.

1. A system for optimizing an implant position in an anatomical joint of a patient, the system comprising a storage media and a processor, wherein the processor is configured to:
receive medical image data representing a three dimensional image of the joint from the storage media;
obtain a three dimensional virtual model of the joint which is based on the received medical image data;
identify damage in the joint based on the received medical image data and/or the three dimensional virtual model of the joint;
select a suitable implant template based on the identified damage; and
position the selected implant template in the three dimensional virtual model of the joint; wherein the processor is configured to position the implant template by:
placing the implant template so that at least a major part of the damage is covered; and
optimizing the tilt of the implant axis in order to minimize the total penetration into the bone.

2. A system according to claim 1, wherein the processor is configured to place the implant template so that the implant hat will at all points be thick enough to ensure mechanical stability, and preferably also thick enough to ensure firm anchoring towards cartilage and bone.

3. A system according to claim 1, wherein the processor is configured to place the implant template so that the implant hat at each point of its circumference penetrates at least a predetermined minimum depth Dₘᵢₙ into the bone.

4. A system according to claim 1, wherein the processor is configured to optimize the tilt of the implant axis by minimizing the maximum penetration depth Dₘₐₓ into the bone along the circumference of the implant hat.

5. A system according to claim 1, wherein the processor is configured to optimize the tilt of the implant axis by minimizing the total volume of bone and/or cartilage to be removed for implanting the implant.

6. A system according to claim 1, wherein the processor is configured to optimize the tilt of the implant axis by minimizing the surface area of the implant penetration into the bone.

7. A system according to claim 1, wherein the processor is configured to restart the optimization of the implant position if at least one predetermined demand on the implant design is not fulfilled.

8. A system according to claim 7, wherein said at least one predetermined demand on the implant design is that the implant hat must have a predetermined minimum thickness Tₘᵢₙ.

9. A method of optimizing an implant position in an anatomical joint of a patient, the method comprising:
receiving medical image data representing a three dimensional image of the joint;
obtaining a three dimensional virtual model of the joint which is based on the received medical image data;
identifying damage in the joint based on the received medical image data and/or the three dimensional virtual model of the joint;
selecting a suitable implant template based on the identified damage; and
positioning the selected implant template in the three dimensional virtual model of the joint; wherein the positioning involves:
placing the implant template so that at least a major part of the damage is covered; and
optimizing the tilt of the implant axis in order to minimize the total penetration into the bone.

10. A method according to claim 9, wherein the positioning involves placing the implant template so that the implant hat will at all points be thick enough to ensure mechanical stability, and preferably also thick enough to ensure firm anchoring towards cartilage and bone.

11. A method according to claim 9, wherein the positioning involves placing the implant template so that the implant hat at each point of its circumference penetrates at least a predetermined minimum depth Dₘᵢₙ into the bone.

12. A method according to claim 9, wherein the optimization of the tilt of the implant axis involves minimizing the maximum penetration depth Dₘₐₓ into the bone along the circumference of the implant hat.

13. A method according to claim 9, wherein the optimization of the tilt of the implant axis involves minimizing the total volume of bone and/or cartilage to be removed for implanting the implant.

14. A method according to claim 9, wherein the optimization of the tilt of the implant axis involves minimizing the surface area of the implant penetration into the bone.

15. A method according to claim 9, wherein if at least one predetermined demand on the implant design is not fulfilled, the optimization of the implant position is restarted.

16. A method according to claim 15, wherein said at least one predetermined demand on the implant design is that the implant hat must have a predetermined minimum thickness Tₘᵢₙ.

17. A method according to claim 9, wherein the anatomical joint is a knee, an ankle, a hip, a toe, an elbow, a shoulder, a finger or a wrist.

18. A non-transitory machine-readable medium on which is stored machine-readable code which, when executed by a processor, controls the processor to perform the method steps of claim 9.
